(19) United States
(12) Plant Patent Application Publication
(10) Pub. No.: US 2013/0191954 P1
(43) Pub. Date: Jul. 25, 2013

(54) Sansevieria Plant Named 'Black Jack'

(50) ;

(71) Applicant: Marivn Gonzalez Villegas, San Ramon (CR)

(72) Inventor: Marivn Gonzalez Villegas, San Ramon (CR)

(73) Assignee: FLORICA FARMS, INC.

(21) Appl. No.: 13/374,904

(22) Filed: Jan. 23, 2012

(51) Int. Cl. A01H 5/00

(52) U.S. Cl. PLT/382

(57) ABSTRACT

The new variety Black Jack is part of the “compact sansevieria varieties”. It can reach 40 cm in length, with an average of approximately 8 leaves per plant. The plant is shaped like an inverted pyramid, the arrangement of the leaves is in the form of a rosette. Shows colors very similar to its parent variety (‘Black Gold Superba’), however it doesn't show variegated bands into the margins, the margins have only a yellow solid color, and ‘Black Jack’ presents more leaves per rosette than the parent.

### LATIN NAME OF THE GENUS AND SPECIES OF THE CLAIMED PLANT

Sansevieria trifasciata

### VARIETY DENOMINATION

Black Jack

### BACKGROUND OF THE INVENTION

The present invention relates to a new and distinct cultivar of Sansevieria plant, botanically known as Sansevieria trifasciata, of the family Ruscaceae, hereinafter referred to by the cultivar name ‘Black Jack’.

Sansevieria is a monocotyledonous and succulent, flowering plant which can be produced in either hard-leafed or soft-leafed varieties for interior use as a house plant or outdoor ornamental plant.

The new Sansevieria ‘Black Jack’ originated from a naturally-occurring mutation of the Sansevieria ‘Black Gold Superba’ (unpatented). The new Sansevieria ‘Black Jack’ was discovered and selected by the inventor, Marvin Gonzalez Villegas, as a single flowering plant within the progeny of the stated mutation in a controlled environment in 2005 in Monterrey, San Carlos, Alajuela province, Costa Rica.

Asexual reproduction of the new Sansevieria cultivar by vegetative cuttings was first performed in January 2005 in Monterrey, San Carlos, Alajuela, Costa Rica, and demonstrated that the combination of characteristics as herein disclosed for the new cultivar are firmly fixed and retained through successive generations of asexual reproduction. The new cultivar reproduces true to type.

### BRIEF SUMMARY OF THE INVENTION

The following traits have been repeatedly observed and are determined to be unique characteristics of ‘Black Jack’, which in combination distinguish this Sansevieria as a new and distinct cultivar:

1. Green colored foliage with yellow margins and yellow-green and pale green bands in the leaf blade.

2. Wide and lanceolate leaves (30 cm long) and supported in a rosette.

3. 30-40 cm plant height.

4. Erect Plant habit.

5. Needs little water and is very drought tolerant.

The new variety Black Jack, is considered a variety of Sansevieria in the group denominated as “compact Sansevieria plants”. The new variety presents an average of 7-10 leaves per plant when it reaches its maturity (between 8-10 months). The variety has an erect growth habit and the entire plant can reach a height range between 30 and 40 cm. The leaves are narrow (6.5 cm average measured at the widest part of the mature leaf) and lanceolate, with a soft waxy texture and the blades are thin and slightly wavy. The plant has an inverted triangle shape.

This variety is moderately drought tolerant and adapts to conditions of light and shade (interior and exterior).

The leaf blade is deep yellowish-green colored (RHS 141A), and also presents non-uniform horizontal variegated bands on both sides of some leaves, with Yellow-Green RHS 149D and RHS 149B coloration. In addition, the leaves have irregular margins on both sides with an average width of 1 cm. The margin coloration is brilliant yellowish-green (RHS 154B) and does not have bands into the margins.

Plants of the new Sansevieria ‘Black Jack’ differ from plants of the parental cultivar, ‘Black Gold Superba’ (unpatented) in the characteristics described in Table 1. The variety ‘Black Gold Superba’ gave rise to the var. Black Jack. The ‘Black Gold Superba’ variety is also considered to be part of the “compact Sansevieria group”. ‘Black Gold Superba’ shows less vigor than Black Jack, has fewer leaves per plant (3-4), and an average width of 6.5 cm (measured in the middle of the mature leaf.) The growth habit is erect and presents wide leaves supported in a rosette. The entire plant can reach a size between 30-40 cm, and the plant has, as for ‘Black Jack’, an inverted triangle shape. The leaves are lanceolate with a soft, waxy texture. The younger leaves show a yellowish green vivid coloration (RHS 134A) in the interior of the leaf blade. This area shows also irregular streaks or yellowish-green bands (RHS 142D) and the margins in both sides of the leaf are variegated with irregular horizontal bands that alternate between light yellowish green (RHS 142D) broad bands and brilliant yellowish green (RHS 149A) thin bands. When the leaf is mature, the deep color inside turns yellowish green (RHS 141B), while margins alternate between vivid greenish yellow (RHS 2A) and light-yellowish green (RHS 2C) colors.

TABLE 1
Comparison with Parental Varieties

| | | Female Parent |
|---|---|---|
| | New Cultivar | Black Gold Superba |
| Characteristic | Black Jack | (unpatented) |
| Plant Vigor | more vigorous (more | less vigorous (less |
| | leaves) | leaves) |
| Color | variegated leaves | green leaves with yellow |
| | (medium green cross- | margins. |
| | banding and dark | |
| | green margins) | |
| Shape | wide and lanceolate | wide and lanceolate |
| | leaves on a rosette | leaves |
| Texture of | smooth-textured leaves | smooth-textured leaves |
| leaves | | |

Of the many commercial cultivars known to the present inventor, the most similar in comparison to the new Sansevieria ‘Black Jack’ is the Sansevieria ‘Black Gold Superba’ (unpatented), in the characteristics described in Table 1.

### BRIEF DESCRIPTION OF THE PHOTOGRAPHS

The accompanying photographs illustrate the overall appearance of the new Sansevieria cultivar ‘Black Jack’ showing the colors as true as is reasonably possible with colored reproductions of this type. Colors in the photographs may differ slightly from the color value cited in the detailed botanical description which accurately describe the color of ‘Black Jack’.

FIG. 1 Shows a side view of an eight-month old plant of Sansevieria ‘Black Jack’ in a 21 cm diameter pot.

FIG. 2 Shows a top view of an eight-month old plant of Sansevieria ‘Black Jack’ in a 21 cm diameter pot.

FIG. 3A Shows a close-up view of the upper side of a typical leaf of ‘Black Jack’ taken from an eight-month old plant.

FIG. 3B Shows a close-up view of the lower side of a typical leaf of ‘Black Jack’ taken from an eight-month old plant.

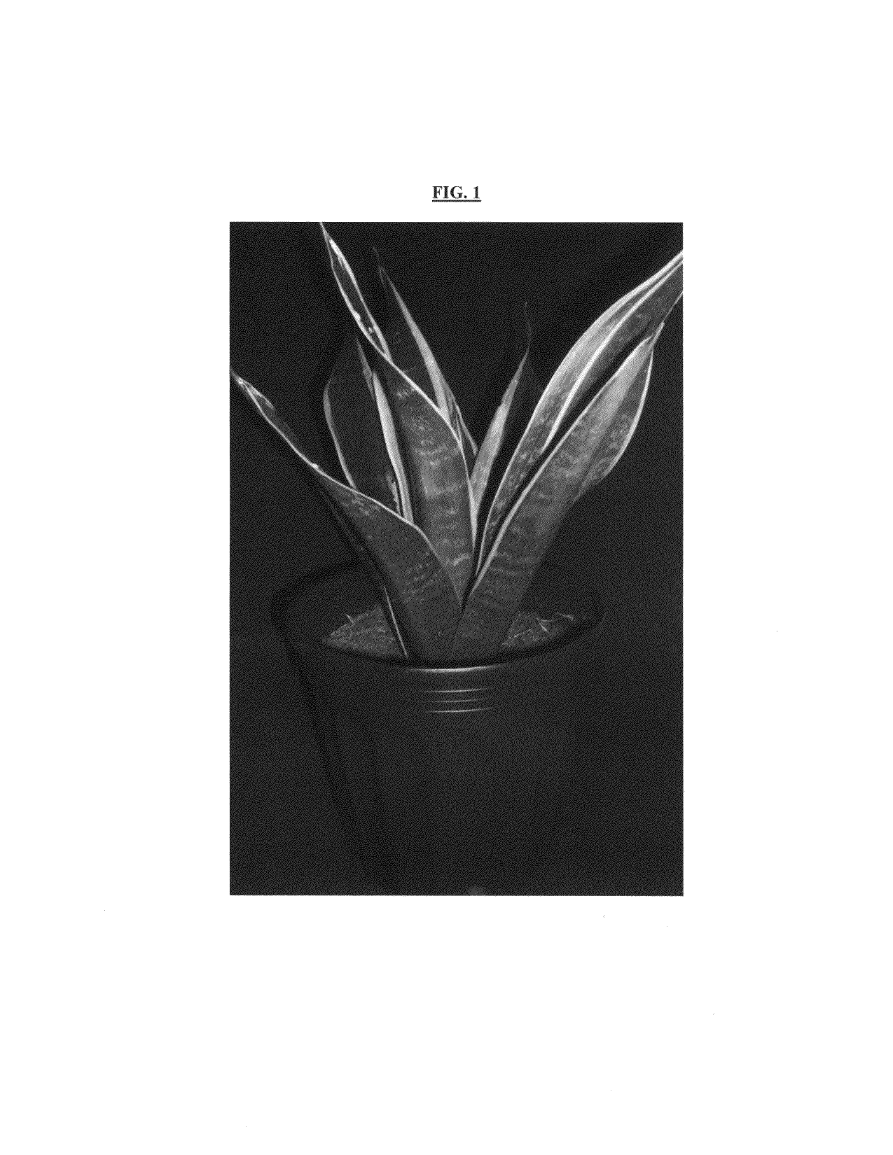

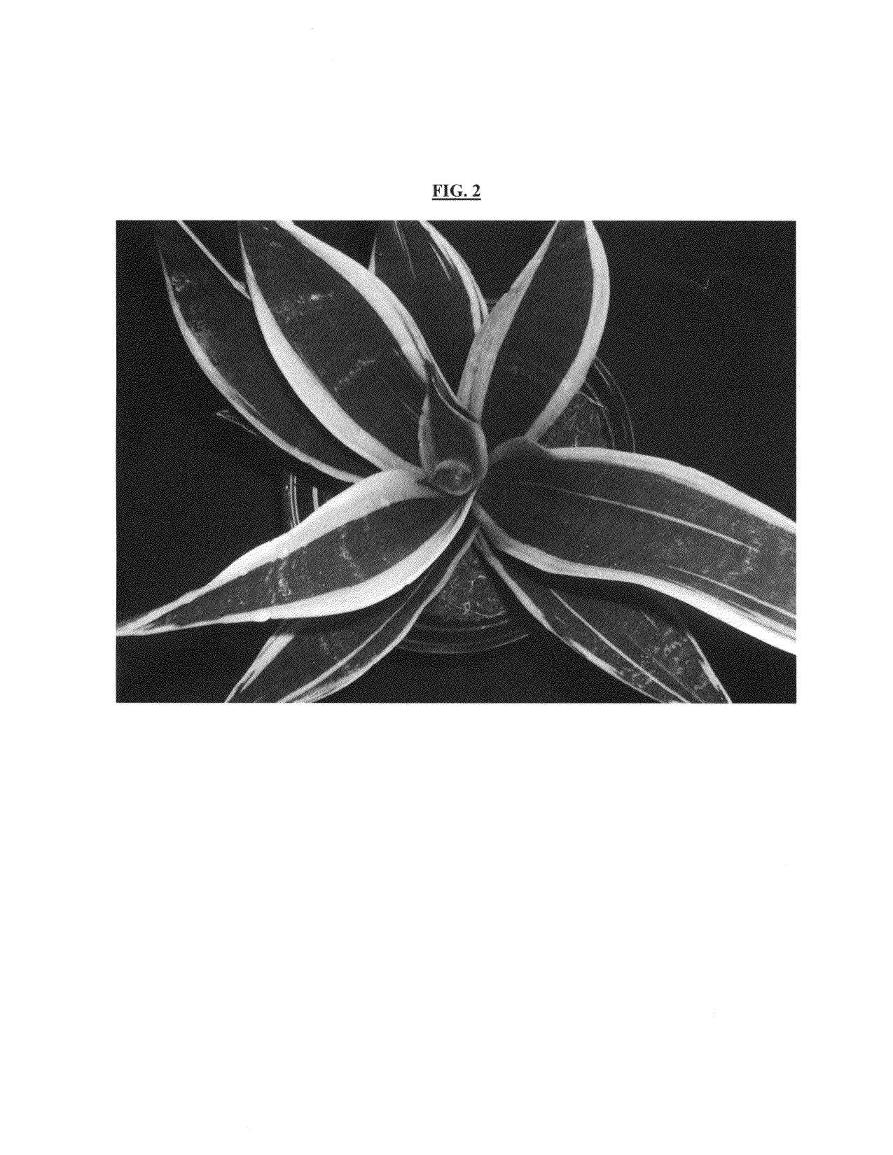

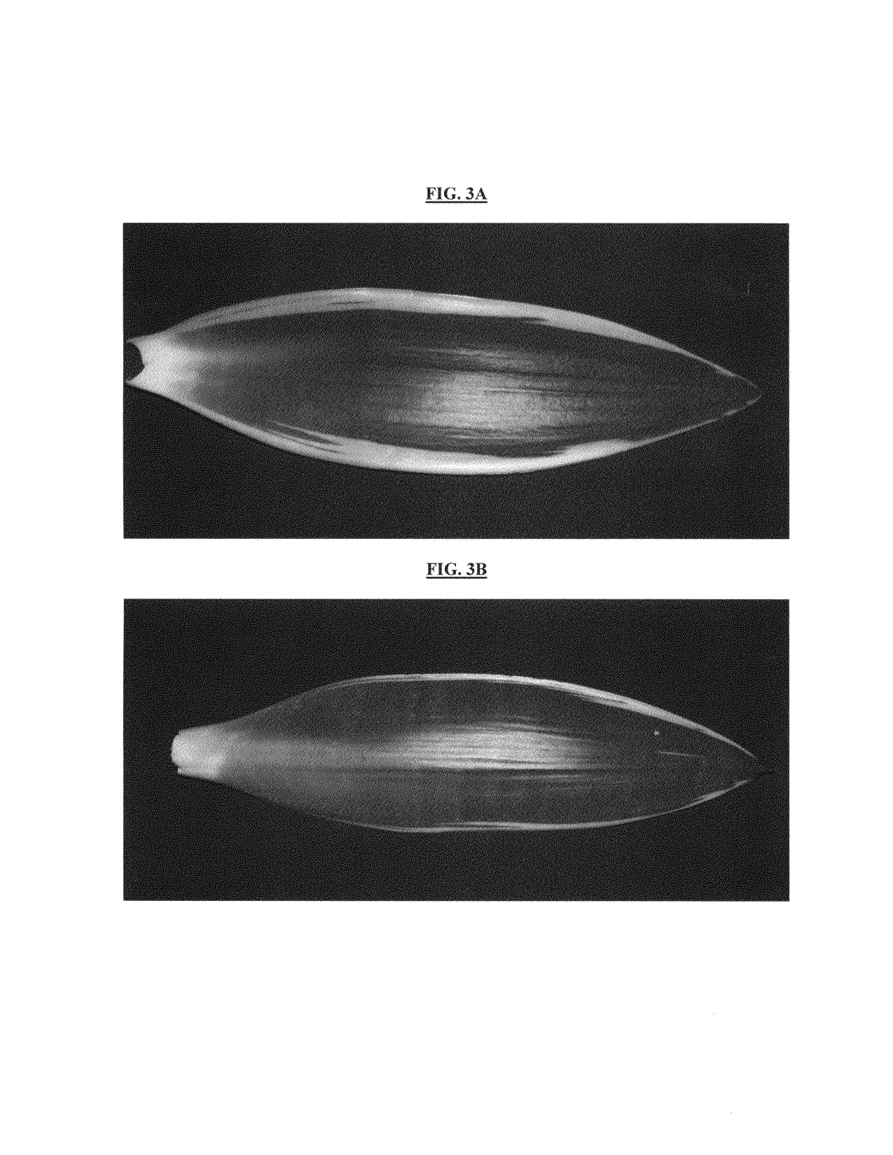

### DETAILED BOTANICAL DESCRIPTION

The new Sansevieria ‘Black Jack’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe the new Sansevieria cultivar ‘Black Jack’ as grown in open-field conditions in Monterrey, San Carlos, Alajuela province, Costa Rica, which closely approximate those generally used in commercial practice. Average temperature at Monterrey is 24.5° C. during the day and 18° C. at night. Plants grow under natural light conditions.

This crop does not require constant fertilization, however, to increase production per area about 200 kg ha⁻¹ N, 150 kg ha⁻¹ of P and 100 kg ha⁻¹ K are required. Production is closely related with soil conditions in which the plants are grown.

Color references were made according to The Royal Horticultural Society Color Chart (R.H.S., April 2009). The photographs and descriptions were made during the rainy season in Monterrey, San Carlos, Alajuela province, Costa Rica, when outdoor day average temperature was 23° C. The age of the plants described is 8 months.
Classification:
   Botanical.—Sansevieria trifasciata.
   Variety denomination.—Black Jack.
Parentage: Sansevieria trifasciata ‘Black Gold Superba’ (unpatented).
Optimal growth conditions:
   Light intensities.—High adaptability to outdoor or indoor conditions.
   Temperature.—Day: 24° C. to 32° C. Night: 18° C. to 23° C.
   Temperature tolerance.—Tolerant to temperatures between 12° C. and 35° C.
   Fertilization.—200, 150 and 100 kg ha⁻¹ of N, P and K.
   Growth regulators.—Not necessary.
Propagation:
   Type.—Vegetative, by rooted cuttings.
   Rooting habit description.—Rhizomes, with short roots.
   Time to initiate roots.—15 to 22 days at 18-25° C.
   Time to produce a rooted cutting.—About 60 to 90 days at 18-25° C.
Plant:
   General appearance and form.—.
   Height.—About 40 cm when grown on the ground; About 35 cm when grown in a 21 cm size container.
   Spread.—About 25 cm when grown on the ground; About 20 cm when grown in 21 cm size container.
   Form.—Monocot; leaf bases arranged in a rosette around growing point.
   Shape.—inverted triangle.
   Growth rate and habit.—About 2 cm per week; upright.
   Fragrance.—none.
   Stem.—Modified (Rhizome and foliage).
   Length.—About 8 cm when grown in ground; About 6 cm when grown in 21 cm size container. Diameter: About 1.7 to 2 cm Shape: round. Texture: rough Color: RHS 17A Strength: strong and durable Internode length: About 0.3 mm to 0.7 mm.
Foliage:
   Quantity.—About 5 leaves.
   Arrangement and attachment.—Single, alternate; leaf bases arranged in a rosette around growing point.
   Leaf length.—About 30 cm.
   Leaf width.—About 6.5 cm.
   Overall shape of leaf.—lanceolate.
   Apex shape.—acute, triangular.
   Base shape.—plane.
   Margin.—sharp, smooth.
   Texture.—Upper Surface: smooth and waxy texture Under Surface: smooth and waxy texture.
   Pubescence.—None.
   Color of mature leaf.—Upper Surface: RHS 141A, 149D, 149B and 154B Lower Surface: RHS 141A, 149D, 149B and 154B.
   Color of immature leaf.—Upper Surface: RHS 141A, 149D, 149B and 154B Lower Surface: RHS 141A, 149D, 149B and 154B.
   Venation.—Pattern: longitudinal/parallel leaf shape Color: None.
   Leaf fragrance.—None.
Inflorescence description:
   Arrangement and type.—Inflorescence.
   Orientation at opening.—out of the spike.
   Quantity.—Per Plant with at least one open flower: One inflorescence per plant About 22 flowers opened 2 weeks after appearance of the spike. Flowers sporadically and profusely under growth conditions.
   Inflorescence size.—Length: About 28 cm Diameter: About 0.4 mm.
   Flower size.—Length: About 2 cm Diameter: About 0.1 mm Depth: About 1 cm.
   Fragrance.—Soft and pleasant.
   Bud.—.
   Rate of opening.—About 7 to 8 days, according to weather.
   Shape.—ovoid.
   Length.—About 22.3 cm.
   Diameter.—About 0.3 mm.
   Color.—Apex: RHS 150A Base: RHS 142A.
   Texture (both surfaces).—smooth.
Petals:
   Quantity.—6.
   Arrangement.—star-shaped.
Reproductive organs:
   Androecium.—Stamen: Number: 5per flower. Length: About 1.3 cm Color: RHS 150C Anther: Length: About 0.2 mm Width: About 0.1 mm Color: RHS 150C Filament: Length: About 1.1 mm Color: RHS 150C Pollen: Amount: moderate Color: RHS 154D.
   Gynoecium.—Pistil: Number: One per flower Length: About 2.1 cm Stigma: Length: About 0.6 mm Width: About 0.8 mm Color: RHS 150C Style: Length: About 1.3 mm Color: RHS150C Ovary: Length: About 3 mm Width: About 2.5 mm Color: RHS 150C.
Weather resistance: Drought tolerant
Disease/pest resistance: not observed for disease/pest resistance.
Disease/pest susceptibility: not observed for disease/pest susceptibility.

I claim:

1. A new and distinct variety of Sansevieria plant named ‘Black Jack’, substantially as illustrated and described herein.
